(19) United States
(12) Patent Application Publication
(10) Pub. No.: US 2026/0060845 A1
(43) Pub. Date: Mar. 5, 2026

(54) WIRELESS MICROFLUIDIC SMART BANDAGE FOR EFFICIENT WOUND EXUDATE MANAGEMENT AND ANALYSIS IN HUMAN SUBJECTS

(71) Applicant: CALIFORNIA INSTITUTE OF TECHNOLOGY, Pasadena, CA (US)

(72) Inventors: Wei Gao, Pasadena, CA (US); Canran Wang, Pasadena, CA (US)

(21) Appl. No.: 19/321,059

(22) Filed: Sep. 5, 2025

Related U.S. Application Data
(60) Provisional application No. 63/690,911, filed on Sep. 5, 2024.

(51) Int. Cl. A61F 13/0203; A61F 13/00; A61F 13/02; A61L 15/18; A61L 15/20; B33Y 40/20; B33Y 80/00

(52) U.S. Cl. CPC A61F 13/0223; A61F 13/00063; A61F 13/0289; A61L 15/18; A61L 15/20; B33Y 40/20; B33Y 80/00; A61F 2013/00089

(57) ABSTRACT

A wearable wound management system integrating a flexible microfluidic assembly and real-time electrochemical sensing to autonomously sample, transport, and analyze wound exudate. A Janus membrane, formed by selective deposition of perfluoroalkyl-functionalized silica nanoparticles and O₂ plasma etching on a PET film, may collect fluid via its superhydrophobic wound-facing side and deliver it to a curved, wedge-shaped microfluidic channel that enhances capillary flow. Downstream, a graded PDMS micropillar array refreshes a sensing region by unidirectional fluid movement. A drop-on-demand inkjet-printed, CO₂ laser-patterned flexible sensor patch may measure nitric oxide, oxygen, hydrogen peroxide, pH, temperature, and other relevant metrics. An encapsulated wireless electronic module may transmit health data for wireless monitoring. This system, combined with machine-learning analytics, may enable continuous, in situ monitoring and predictive wound classification, supporting proactive and personalized chronic wound care.

### CROSS-REFERENCE TO RELATED APPLICATIONS

This application claims the benefit of U.S. Provisional Application No. 63/690,911 filed on Sep. 5, 2024, the contents of which are incorporated herein by reference in their entirety.

### STATEMENT REGARDING FEDERALLY SPONSORED R&D

This invention was made with government support under Grant No(s). HL155815 & DK132666 awarded by the National Institutes of Health. The government has certain rights in the invention.

### TECHNICAL FIELD

The present disclosure relates to wound monitoring and, in some embodiments, to monitoring wound exudate with a wearable wound management system.

### BACKGROUND

Chronic wounds present a significant challenge in clinical care due to their complex etiology and the multifactorial nature of impaired healing. Patients suffering from diabetic ulcers, venous leg ulcers, or pressure sores often experience a prolonged inflammatory phase alongside compromised tissue perfusion. This abnormal wound environment often results in persistent exudate, an imbalance in moisture, and a heightened risk of bacterial infection. The high economic burden and significant impact on patient quality of life further underscore the urgent need for effective wound management strategies. Over the years, standard care approaches such as gauze dressings, absorbent pads, and manual debridement have demonstrated limited effectiveness in both controlling wound moisture and providing timely diagnostic indicators, often leading to suboptimal clinical outcomes.

Wound exudate, the fluid secreted from chronic wounds, may be a rich source of biomolecular information that reflects the dynamic biochemical conditions of the wound bed. It can contain a diverse array of substances including proteins, cytokines, cells, and reactive species such as nitric oxide (NO), hydrogen peroxide (H₂O₂), and dissolved oxygen (O₂). These biomarkers have the potential to provide real-time insights into the wound healing process, particularly in relation to inflammation, infection, and tissue regeneration. However, capturing and analyzing this biofluid in situ poses significant challenges due to its low secretion rate, typically measured in microliters per square centimeter per hour, and the difficulty in isolating new, uncontaminated streams of exudate from the mixture of old fluid that accumulates over time. This intermixing can lead to inaccuracies in biomarker measurements, thereby hindering early intervention and precise wound assessment.

The reactive nature of key analytes such as NO and H₂O₂ adds an extra layer of complexity to wound analysis. These molecules are relevant signaling agents that may facilitate various aspects of wound healing, including microbial defense, angiogenesis, and cellular communication. However, their highly transient nature means that even slight delays in sampling or variations in fluid collection methods can result in rapid oxidation or degradation, ultimately skewing sensor outputs and reducing the reliability of real-time monitoring. Achieving accurate and reproducible measurements often requires that the exudate be rapidly collected, directed to sensors, and replaced in a manner that minimizes stagnation and contaminant mixing. Additionally, environmental factors like pH and temperature can influence both the chemistry of the wound exudate and the stability of the detection mechanisms, making simultaneous and continuous monitoring of these parameters an important aspect of effective wound diagnostics.

### BRIEF SUMMARY

Various embodiments of the present disclosure may discuss a wearable wound management system that may include a Janus membrane having a wound-facing superhydrophobic surface and an opposing superhydrophilic surface, a wedge-shaped microfluidic channel in fluid communication with the Janus membrane, and an array of micropillars. The array of micropillars may include a series of pillars capable of transferring fluid vertically from the wedge-shaped microfluidic channel to an upper gradient pillar array. The array of micropillars may have a gradient of pillar heights arranged downstream of the wedge-shaped microfluidic channel. A sensor patch laminated to the wedge-shaped microfluidic channel may include a flexible substrate and a plurality of electrochemical sensors printed thereon, while a wireless electronic module may be electrically coupled to the sensor patch and encapsulated in a biocompatible elastomer to transmit electrochemical sensor data via Bluetooth. Together, the Janus membrane, wedge-shaped microfluidic channel, array of micropillars, sensor patch and wireless electronic module may define a flexible microfluidic assembly.

The Janus membrane formation may be formed from selective deposition of chemically modified silica nanoparticles and O₂ plasma etching on a polyethylene-terephthalate film, the chemically modified silica nanoparticles may include 1H,1H,2H,2H-perfluorooctyltriethoxysilane-coated silica. The plurality of electrochemical sensors may include a voltammetric nitric oxide sensor, a voltammetric oxygen sensor, an amperometric hydrogen peroxide sensor, a potentiometric pH sensor, and a resistive temperature sensor. The wedge-shaped microfluidic channel may feature a width increasing in a direction away from the Janus membrane and a curvature configured to increase capillary pressure. The array of micropillars may include pillars fabricated from cured polydimethylsiloxane with heights ranging from 0.2 mm to 0.8 mm. The sensor patch manufacturing process may involve drop-on-demand inkjet printing of conductive inks followed by CO₂ laser patterning.

The microfluidic module for wearable wound exudate handling may include an inlet layer with a Janus membrane featuring laser-patterned micropores and opposing hydrophobic/hydrophilic surfaces; a transport layer having at least one wedge-shaped microfluidic channel defined in a biocompatible tape in fluid communication with the inlet layer; and an outlet layer comprising a three-dimensional graded micropillar array molded in polydimethylsiloxane, the outlet layer being in fluid communication with the transport layer. The Janus membrane surface may include a superhydrophobic surface bearing perfluoroalkyl-functionalized silica coating and a superhydrophilic surface. The wedge-shaped channel may span 2-8 mm in length with a maximum width of approximately 0.8 mm and a minimum width of approximately 0.2 mm. The three-dimensional graded micropillar array may form capillary pressure gradients configured to direct fluid away from the outlet layer, may include pillars of at least three discrete heights, and the transport layer may present a hydrophilic channel inside and hydrophobic edges defined by O₂ plasma etching. An alignment feature for lamination to a flexible sensor substrate may also be included.

The method for wearable wound exudate collection and analysis may involve mounting a wearable device on a wound site, the wearable device included the discussed microfluidic module stacked with a flexible electrochemical sensor patch and a wireless electronics module; receiving wound exudate through the Janus membrane within the microfluidic module; transporting the wound exudate through the wedge-shaped channel to a sensing reservoir; refreshing the sensing reservoir by passing the wound exudate through the graded micropillar array; electrochemically sensing the wound exudate to measure at least one reactive species and one environmental parameter via the electrochemical sensor patch; and wirelessly transmitting measurement data representative of electrochemically sensing the wound exudate with the wireless electronics module. Characterizing nitric oxide, oxygen and hydrogen peroxide concentrations by differential pulse voltammetry and amperometry, calibrating the flexible electrochemical sensor patch using sensed pH and temperature, refreshing the sensing reservoir via unidirectional fluid movement out of the reservoir through the graded micropillar array and analyzing the transmitted measurement data with a machine-learning model to predict wound classification or healing time also form part of the method.

Other features and aspects of the disclosed technology will become apparent from the following detailed description, taken in conjunction with the accompanying drawings, which illustrate, by way of example, the features in accordance with embodiments of the disclosed technology. The summary is not intended to limit the scope of any inventions described herein.

### BRIEF DESCRIPTION OF THE SEVERAL VIEWS OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a diagram showing a perspective view of an example wearable wound management system, in accordance with various embodiments of the disclosed technology.

FIG. 2A is a diagram showing a perspective view of exemplary electrochemical sensors, in accordance with various embodiments of the disclosed technology.

FIG. 2B is a diagram showing an enlarged view of exemplary electrochemical sensors, in accordance with various embodiments of the disclosed technology.

FIG. 3 is a diagram showing an example fabrication process for an example wearable wound management system, in accordance with various embodiments of the disclosed technology.

FIG. 4 is a diagram showing an example fabrication process for an array of micropillars, in accordance with various embodiments of the disclosed technology.

FIG. 5 is an example circuit diagram for an example wearable wound management system, in accordance with various embodiments of the disclosed technology.

FIG. 6 is a diagram showing an exemplary method for wearable wound exudate collection and analysis, in accordance with various embodiments of the disclosed technology.

FIG. 7 is a diagram showing an example flexible microfluidic assembly incorporating a Janus membrane, wedge-shaped channel, and graded micropillar array, in accordance with various embodiments of the disclosed technology.

FIG. 8 is a diagram showing a perspective view of an array of example wearable wound management systems mass-produced via inkjet printing, in accordance with various embodiments of the disclosed technology.

FIG. 9 is a diagram showing an expanded view of an example flexible microfluidic assembly, in accordance with various embodiments of the disclosed technology.

FIG. 10 is a diagram showing an exemplary Janus membrane inlet for exudate collection and unidirectional transport of fluids, in accordance with various embodiments of the disclosed technology.

FIG. 11 is a diagram showing various perspective views of an example array of micropillars, in accordance with various embodiments of the disclosed technology.

FIG. 12 is a diagram showing an example method of using an example wearable wound management system on a human patient in the clinical setting for wound assessment, in accordance with various embodiments of the disclosed technology.

The figures are not intended to be exhaustive or to limit the invention to the precise form disclosed. It should be understood that the invention can be practiced with modification and alteration, and the disclosed technology be limited only by the claims and the equivalents thereof.

### DETAILED DESCRIPTION

Embodiments of the present disclosure generally relate to wearable wound management systems that incorporate advanced microfluidic modules and flexible sensor technologies, and more particularly to systems designed to address the challenges associated with continuous, in situ analysis of wound exudate. The disclosed technology provides an improved platform for more efficient wound exudate collection, transport, and refreshing alongside real-time biochemical sensing.

In recent years, microfluidic approaches have emerged as a potential solution to the challenges of wound exudate management and analysis. By leveraging intricate channel architectures and surface energy modifications, microfluidic devices can guide small volumes of fluid in a controlled and pump-free manner. Such systems may utilize capillary forces, which can be enhanced by specially engineered geometries such as wedge-shaped channels and graded micropillar arrays. These geometric features may be designed to facilitate unidirectional fluid flow and to promote efficient sampling, ensuring that fresh exudate is rapidly isolated from the wound bed while effectively flushing away any residual or mixed fluid. This capability is important to maintaining the integrity of the biomarker analysis, as the isolation of fresh fluid minimizes potential errors due to the mixing of older exudate that may carry degraded or altered biochemical signatures.

The microfluidic elements of the system may be further enhanced through the employment of a Janus membrane that can exhibit dual hydrophobicity. One surface of the Janus membrane, which may face the wound bed, may be engineered to be superhydrophobic, thereby repelling the wound exudate initially. The opposite surface may be engineered to be superhydrophilic, which may facilitate the rapid uptake and passage of wound exudate through laser-patterned micropores. This selective surface treatment, which may be achieved through the deposition of chemically modified silica nanoparticles and specialized O₂ plasma etching on a polyethylene terephthalate (PET) substrate, can create a strong driving capillary force for the fluid to transition from one surface to the other in a controlled manner. The Janus membrane may play an initial role in directing wound exudate into the subsequent microfluidic channels while preserving the sensitivity and accuracy of the sensing components downstream.

Beyond the collection of fluid at the wound bed, the microfluidic system may include a wedge-shaped channel with a geometry that increases in width as it extends away from the Janus membrane. This structural design may ensure that the capillary forces are optimized to pull the fluid away from the wound bed and into the channel, and further enhances the unidirectional flow of the collected wound exudate. A wedge-shaped microfluidic channel, with its carefully engineered dimensions (for example, channels ranging from approximately 0.2 mm to 0.8 mm in width and extending from 2 mm to 8 mm in length), may create a gradient of capillary pressure that augments the automatic flow of fluid through the system. This continuous transport mechanism may be relevant to overcoming the limitations imposed by the low rates of wound exudate secretion, typically in the microliter range, and ensures that the monitored wound exudate remains representative of current wound conditions.

Further relevant to the efficient management of wound exudate may be the incorporation of a three-dimensional, graded micropillar array located downstream of the wedge-shaped channel. The micropillars may be fabricated from cured polydimethylsiloxane (PDMS) and exhibit a gradient of heights, for instance, ranging from 0.2 mm to 0.8 mm, to establish capillary pressure gradients that drive unidirectional wound exudate movement away from the sensor array (as discussed below). This structural arrangement may allow for the automatic refreshing of the sensing reservoir by directing old or spent (previously sensed) wound exudate away from the active sensing region and replacing it with newer exudate from the wound site. For embodiments without a sensing reservoir, the graded micropillar array may direct wound exudate away from the electrochemical sensors that wound exudate passes over after being channeled by the wedge-shaped channel. The geometry and arrangement of the micropillars may be optimized to minimize fluid mixing and to maintain the temporal resolution necessary for accurate detection of rapidly fluctuating biomarkers, thereby ensuring that the sensor readings accurately reflect the current biochemical status of the wound.

In addition to the advanced microfluidic fluid management system, the present disclosure may incorporate a flexible sensor patch that may be integrated into the wearable assembly. Embodiments of the sensor patch may be manufactured on a flexible substrate using a combination of drop-on-demand inkjet printing and CO₂ laser patterning. The resulting electrochemical sensors may be engineered for the analysis of critical wound biomarkers, including reactive species such as nitric oxide (NO), hydrogen peroxide (H₂O₂), and dissolved oxygen (O₂), in addition to monitoring pH and temperature. Further embodiments may include other wound biomarkers such as creatinine and cytokines. Each sensor may be patterned to optimize sensitivity and to reduce interference from other electroactive species present in complex wound exudate matrices. The sensors may be capable of performing voltammetric and amperometric measurements (along with other relevant measurements) under continuous, real-time operation, thereby providing a detailed and dynamic profile of the wound environment.

The integration of flexible electronics within the system may improve the functionality by enabling wireless transmission of real-time measurement data. A wireless electronic module, which may be encapsulated in a biocompatible elastomer, can be electrically coupled to the sensor patch and configured to transmit chemical sensor data using protocols such as Bluetooth. This wireless communication may allow for remote monitoring and data analysis, facilitating continuous oversight of wound healing without necessitating frequent clinical visits. The more immediate feedback provided by the wireless module may ensure that emerging complications such as early infection or inefficient exudate management can be rapidly identified and addressed, improving patient outcomes.

Challenges associated with conventional clinical assessment methods for wound evaluation may be addressed by the continuous, real-time monitoring capabilities of the present system. Traditional diagnostic approaches, which often rely on manual inspections and intermittent measurements on a limited scale, often miss subtle variations in wound biomarkers. The lack of objective, quantified data has necessitated the development of solutions capable of continuous sampling and analysis. By coupling present microfluidic management system with advanced electrochemical sensing, the present technology may overcome these limitations and deliver a more constant stream of reliable data. This continuous data stream is not only relevant for detecting minute changes in the wound environment but also serves as a robust source of information for advanced analytical techniques, such as machine learning, which can be used to predict wound classification and healing time.

Manufacturing of both the microfluidic modules and the sensor patches may leverage advanced printing and patterning techniques that are more scalable and cost-effective. Processes such as drop-on-demand inkjet printing allow for the high-throughput deposition of conductive inks and chemically active materials on flexible substrates, while CO₂ laser patterning may allow for the precision necessary to define intricate microfluidic channels and sensor geometries. The convergence of these fabrication methods may result in a wearable assembly that is robust and flexible, as well as sufficiently durable to withstand the dynamic environment of chronic wound care over extended periods. This manufacturing approach ultimately may enable the production of devices that are both reproducible and economically viable for widespread clinical application.

Furthermore, the system may be designed with an emphasis on biocompatibility and user comfort, taking into account the sensitive nature of chronic wound environments. Materials selected for the construction of the device-ranging from the PET substrate of the Janus membrane to the PDMS used for micropillar arrays and sensor encapsulation-may be chosen for their improved biocompatibility and minimal inflammatory response. This selection of materials may allow the wearable system to be used continuously without adverse effects on the wound healing process.

The integration of multiple functional components-namely, the microfluidic fluid handling module, the flexible sensor patch, and the wireless electronic module-into a single, cohesive wearable assembly may represent an advancement in wound care management. Each component may be engineered to perform its function in concert with the others, resulting in a system that may be capable of continuously monitoring and managing wound exudate in real time. This integrated approach allows for the collection of precise biochemical data that can direct more immediate clinical intervention and feed into advanced analytical frameworks to predict longer-term wound outcomes.

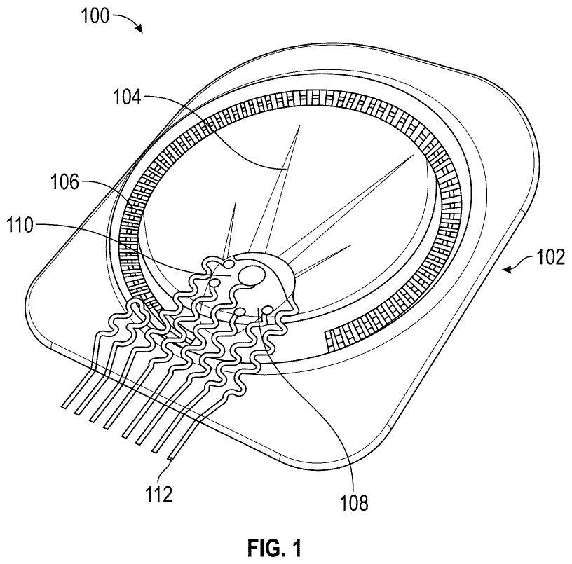

Turning now to the figures, FIG. 1 is a diagram showing a perspective view of an example wearable wound management system 100, in accordance with various embodiments of the disclosed technology. The wearable wound management system 100 may include multiple layers and may be a pump-free microfluidic assembly. The wearable would management system 100 may include a Janus membrane 102, a wedge-shaped microfluidic channel 104, an array of micropillars 106, an electrochemical sensor patch 108, a flexible substrate 110, and one or more sensor terminals 112.

The Janus membrane 102 may be disposed directly on the wound bed. The Janus membrane 102 may be fabricated by selective deposition of perfluoroalkyl-functionalized silica nanoparticles on a polyethylene-terephthalate (PET) film and O₂ plasma etching. Embodiments of the Janus membrane 102 may include a superhydrophobic wound-facing surface that prevents backflow, and an opposing superhydrophilic surface that rapidly draws in wound exudate. Fluid entering the Janus membrane 102 may be directed into a wedge-shaped microfluidic channel 104 in biocompatible tape. The wedge-shaped microfluidic channel 104 may feature a gradually increasing width (≈0.2 mm at the inlet to ≈0.8 mm at the outlet) and a gentle curvature chosen to maximize LaPlace pressure and drive capillary flow.

Superhydrophobicity and superhydrophilicity may describe extreme wetting behaviors of solid surfaces as quantified by the water contact angle. A superhydrophobic surface repels water so strongly that sessile droplets form nearly spherical beads, with static contact angles typically above 150° and very low roll-off angles. In practice, this effect arises from a combination of low-surface-energy coatings (e.g., fluorinated silanes) and hierarchical micro-/nano-scale roughness that traps air beneath the liquid, minimizing solid-liquid contact. By contrast, a superhydrophilic surface attracts water so strongly that droplets spread into thin, uniform films, exhibiting static contact angles below 10°. Achieved through high-surface-energy coatings or plasma treatments (e.g., oxidized silica or titania nanoscale layers), superhydrophilicity maximizes fluid spreading and capillary action. Together, these opposing surface extremes enable engineered fluid control in applications ranging from self-cleaning coatings to passive microfluidic channels.

Downstream of the microfluidic channel 104, an array of micropillars 106 may be molded in cured polydimethylsiloxane (PDMS) and define a three-dimensional, graded structure with discrete pillar heights ranging from 0.2 mm to 0.8 mm. Further embodiments may include pillar heights outside of this range. The array of micropillars 106 may perform continuous wound exudate refreshing by generating a capillary pressure gradient that lifts residual fluid vertically out of the sensing region, preventing mixing of old and fresh wound exudate.

Laminated to the base of wedge-shaped microfluidic channel 104 may be a sensor patch 108 that can carry a flexible substrate 110 printed with an array of electrochemical sensors. The flexible substrate 110 may be produced by drop-on-demand inkjet printing of gold and carbon conductive inks followed by CO₂ laser patterning. The flexible substrate 110 may support multiple sensor terminals 112. Each terminal 112 providing a low-impedance connection between individual working/counter/reference electrodes (e.g., voltammetric NO sensor, voltammetric O₂ sensor, amperometric H₂O₂ sensor, potentiometric pH sensor, resistive temperature sensor) and overlying electronics.

Electrical traces from sensor terminals 112 may route to a wireless electronic module (not shown) encapsulated in biocompatible elastomer. Together, the Janus membrane 102, the wedge-shaped microfluidic channel 104, the array of micropillars 106, the sensor patch 108 with flexible substrate 110, and the sensor terminals 112 may define the conformable, wearable wound management system 100 capable of autonomous exudate handling and real-time biochemical monitoring without obstructing patient mobility.

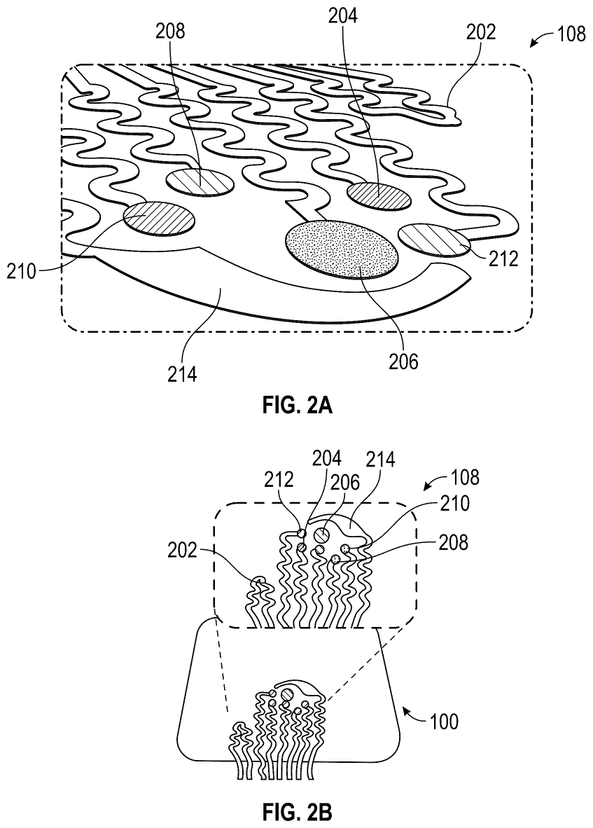

FIG. 2A is a diagram showing a perspective view of exemplary electrochemical sensors disposed on a sensor patch 108, in accordance with various embodiments of the disclosed technology. As illustrated, a flexible, multiplexed electrochemical sensor patch 108 may integrate a plurality of printed electrochemical sensors. For example, the sensor patch 108 may include a temperature sensor 202, a pH sensor 204, a nitric oxide (NO) sensor 206, an oxygen (O₂) sensor 208, and a hydrogen peroxide (H₂O₂) sensor 210, among other biochemically relevant sensors. The electrochemical sensors may be disposed on and supported by a flexible substrate (e.g., flexible substrate 110). The sensor patch 108 may also include at least one counter electrode 212 and a printed reference electrode 214. The electrodes, interconnects, and contact pads may be patterned on the substrate using additive printing techniques and selective overcoats, such that the active sensing areas are exposed to biological fluid while the remainder of the conductors are encapsulated by dielectric passivation.

In various embodiments, the flexible substrate may be a polymeric film such as polyethylene terephthalate (PET) having a thickness between approximately 25 micrometers and 125 micrometers. Conductive features may be formed by drop-on-demand inkjet printing of metallic and/or carbon inks onto the substrate, followed by thermal curing. Channel openings and/or sensor windows can be further defined by CO₂ laser patterning. A printed or transferred electroactive layer may be subsequently applied to form the sensing chemistries for each target analyte as discussed herein. An insulating overcoat (e.g., a styrene-butadiene copolymer) may define apertures over each electrode's active area and protects the remaining traces. The sensor patch 110 may include alignment fiducials to facilitate lamination beneath a microfluidic transport layer and around a sensing reservoir. The combination of each electrode's active may define the sensing region. In some embodiments, the sensing region may be disposed within a sensing reservoir.

Temperature sensor 202 may be a resistive temperature detector patterned from printed carbon or other resistive ink in a serpentine or meander geometry to increase path length and sensitivity. The temperature sensor 202 may be configured for up to two- or four-wire readout and exhibit a substantially linear change in resistance with temperature over a physiologic range (for example, approximately 25 to 45 C). Representative baseline resistances can range from approximately 0.5 kΩ to approximately 50 kΩ depending on line width, thickness, and geometry. Temperature sensor 202 may provide real-time temperature compensation for the electrochemical measurements and also may serve as an environmental parameter indicative of wound status.

pH sensor 204 may be a potentiometric electrode comprising a noble metal underlayer (e.g., inkjet-printed gold) over which a pH-sensitive conducting polymer film, such as polyaniline, may be electropolymerized. In use, the pH sensor 204 may be measured at open-circuit potential relative to the shared reference electrode 214. The sensor output may vary monotonically with hydrogen ion activity in the wound fluid, typically exhibiting a near-Nernstian slope across physiologic pH values (e.g., pH 5 to pH 8). The pH readout may be utilized both as a clinical parameter and for on-patch calibration of the redox sensors.

NO sensor 206 may include a working electrode based on a high-surface-area carbonaceous electrode modified with a selective NO recognition layer. In certain embodiments, the working electrode may comprise a laser-engraved graphene (LEG) or similar mesoporous carbon electrode onto which a graphene oxide dispersion may be applied to improve bioreceptor immobilization and charge transfer. A hemoglobin (Hb) layer may be immobilized at the electrode surface as a selective reductive catalyst for NO, and a permselective overcoat (e.g., a thin Nafion film) may be applied to mitigate fouling and exclude interfering anions and larger electroactive species. During operation, NO may be quantified by voltammetric reduction (e.g., differential pulse voltammetry) at a negative potential versus the reference electrode 214, producing a concentration-dependent peak current. This configuration may promote selective NO detection in the presence of electroactive interferents commonly present in wound exudate.

O₂ sensor 208 may include a working electrode formed from a printed gold layer, and in some embodiments, comprising sintered gold nanoparticles to increase electroactive surface area and catalytic activity. A thin protective and/or permselective membrane can be applied to stabilize the electrode and moderate mass transport. Dissolved oxygen may be quantified by measuring the reduction response (e.g., via differential pulse voltammetry) at a potential negative of the open-circuit potential but less negative than the NO reduction potential, enabling multiplexed operation without crosstalk. The O₂ signal may provide an indicator of local perfusion and tissue oxygenation at the wound site.

H₂O₂ sensor 210 may comprise a catalytic redox couple deposited onto a noble metal underlayer at the working electrode. In certain embodiments, Prussian blue (PB) may be electrodeposited onto the gold or carbon electrode to serve as an efficient electrocatalyst for peroxide reduction at low overpotential. To enhance operational stability during extended exposure to complex wound matrices, a nickel hexacyanoferrate (NiHCF) layer can be co-deposited or over-deposited on the PB film. A permselective coating (e.g., Nafion) further may improve antifouling performance. The H₂O₂ sensor may be operated amperometrically at a fixed potential of approximately 0 V versus reference electrode 214, producing a current proportional to peroxide concentration.

Each sensing element may utilize at least one working electrode tailored to the target analyte's redox chemistry. The working electrodes can be round, rectangular, or interdigitated geometries, with exposed active areas sized, for example, between approximately 0.2 mm and 3 mm in characteristic dimension. Where advantageous, multiple working electrodes may be provided for a given analyte to allow ratiometric measurements, redundancy, or different dynamic ranges. Conductive traces routed from each working electrode to a connector region may be covered by a passivation layer to preserve flexibility and biocompatibility.

Reference electrode 214 may be a printed quasi-reference electrode rendered into a stable Ag/AgCl reference by depositing silver onto a printed gold seed, followed by chloridization. For example, silver may be electroplated onto a printed gold pad and chemically or electrochemically converted to Ag/AgCl, after which a reference membrane may be applied (e.g., a polyvinyl butyral matrix containing a chloride salt) to maintain a stable chloride environment and minimize drift. Reference electrode 214 may be positioned on the sensor patch 108 to be co-located with the microfluidic sensing reservoir such that it is wetted by the same wound exudate as the working electrodes during operation.

Counter electrode 212 may be formed from a printed gold, platinum, or carbon region, optionally enlarged to reduce polarization during amperometric or voltammetric measurements. All electrodes may be connected via printed interconnects (e.g., terminals 112) to contact pads configured for attachment to, or direct soldering/bonding with, a wireless electronic module. The interconnect metallization and contact pads may be reinforced or overprinted to increase conductivity and mechanical robustness.

The active sensing areas of temperature sensor 202, pH sensor 204, NO sensor 206, O₂ sensor 208, and H₂O₂ sensor 210 may be exposed through windows in an encapsulating dielectric. One or more hydrophilic surface treatments may be applied at the sensing windows to promote uniform wetting by wound exudate delivered by the overlying microfluidics. The sensors can be arranged circumferentially around a central opening or reservoir aligned with the microfluidic outlet so that all sensors contact refreshed fluid. In some embodiments, the total footprint of the sensor patch 108 may be between approximately 10 mm by 10 mm and approximately 30 mm by 30 mm, with a bend radius less than approximately 25 mm to accommodate body motion without delamination or signal instability.

During use, the wireless module may sequentially or concurrently actuate the electrochemical sensors according to a programmed schedule. For example, the controller may (i) read temperature sensor 202 to update compensation coefficients, (ii) acquire an open-circuit potential from pH sensor 204 relative to reference electrode 214, (iii) perform differential pulse voltammetry at NO sensor 206 to extract the NO peak current, (iv) perform differential pulse voltammetry at O₂ sensor 208 to extract the oxygen reduction peak, and (v) perform fixed-potential amperometry at H₂O₂ sensor 210. The pH and temperature values may be used to calibrate and correct the reactive-species signals in real time. The shared reference electrode 214 may reduce footprint and support multiplexed, time-staggered measurements without requiring multiple reference elements. Other orders or processes may exist.

While certain embodiments may use inkjet-printed gold and carbon inks, other printable conductors (e.g., silver with appropriate barrier layers, platinum, or conductive polymers) may be employed. The NO biorecognition sensor may use alternative metalloproteins or catalytic films that support selective NO reduction. The O₂ and H₂O₂ electrodes may incorporate different nanostructures or catalysts to tune sensitivity and stability. Alternative permselective and antifouling coatings can be used depending on the intended wear duration and wound environment.

FIG. 2B is a diagram showing an enlarged view of exemplary electrochemical sensors, in accordance with various embodiments of the disclosed technology. In various embodiments, an enlarged view of the wearable wound management system 100 may include a placement of electrochemical sensors on sensor patch 108, as depicted. The sensor patch 108 may carry discrete sensing sites comprising, for example, a temperature sensor 202, a pH sensor 204, a NO sensor 206, an O₂ sensor 208, and a H₂O₂ sensor 210. Each sensing site may include at least one working electrode exposed within a sensing window, while a shared reference electrode 214 may be positioned proximate to, and wetted with, the same fluid as the working electrodes to provide a stable reference during measurements.

In the illustrated arrangement, the sensing sites may be disposed around a central sensing region of the sensor patch 108 aligned with the outlet of the microfluidic transport (e.g., the wedge-shaped channel), such that some or all sensors contact refreshed wound exudate during operation. The temperature sensor 202 may be positioned to sample the same fluid as the electrochemical sensors for real-time compensation. Interconnects and contact pads (not numbered) may be routed toward an edge of the sensor patch 108 for electrical coupling to the wireless module. The enlarged view emphasizes the relative placement of sensors 202, 204, 206, 208, and 210 on patch 108 within system 100; the construction and operation of these sensors can be as described with reference to FIG. 2A.

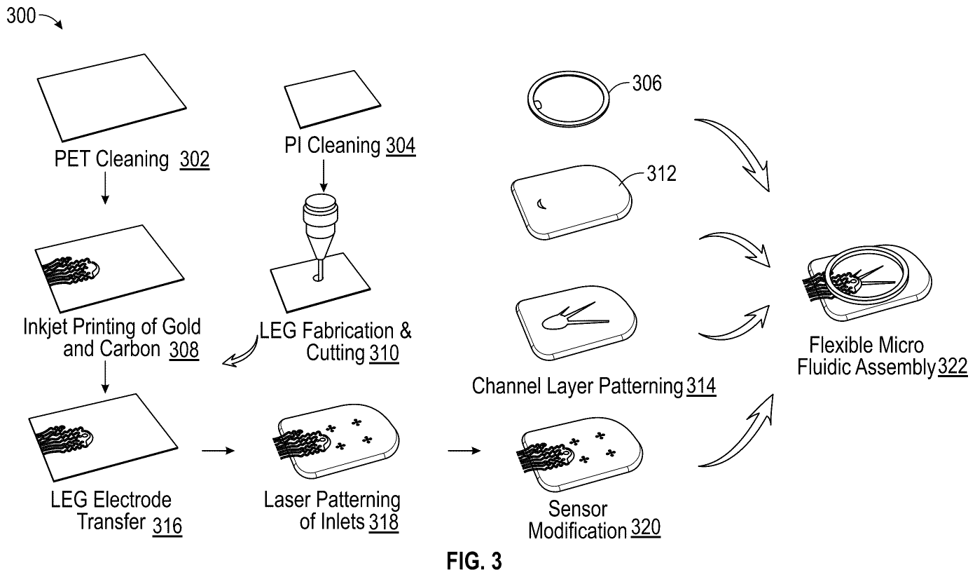

FIG. 3 illustrates an example fabrication process 300 for producing a wearable wound management system that may culminate in a flexible microfluidic assembly 322 (e.g., a wearable wound management system without the wireless electronic module). In general, components may be prepared in parallel and then laminated in registration over a sensor patch to yield a stack that manages wound exudate and supports multiplexed sensing.

Substrate preparation may begin with PET cleaning 302, for example by sequential solvent rinses and/or oxygen plasma to remove residues and increase surface energy for printing and lamination. In parallel, PI cleaning 304 may be performed on a polyimide film destined for laser-engraved graphene (LEG) to promote uniform engraving and subsequent transfer.

Conductive features may be defined by inkjet printing of gold and carbon 308 on the PET substrate, producing electrode underlayers, interconnects, contact pads, and optional counter-electrode regions. Printed features may be thermally cured, and sensor windows can be defined by selective overcoat patterning and/or CO₂ laser ablation to expose the active areas while protecting surrounding traces on the sensor patch.

LEG fabrication & cutting 310 may be carried out on the cleaned PI by CO₂ laser engraving to convert targeted regions into porous, high-surface-area graphene suitable for electrochemical transduction. The engraved LEG may then be singulated to the desired geometry and positioned for LEG electrode transfer 316 onto the printed PET, for example by adhesive- or heat-assisted lamination, aligning LEG working sites with designated sensing windows.

Janus membrane fabrication 312 may proceed on a thin PET or similar film by applying a perfluoroalkyl-functionalized silica nanoparticle coating to create a superhydrophobic surface, while the opposing surface may be rendered superhydrophilic by patterned O₂ plasma etching. Laser patterning of inlets 318 may form through-holes or micropores in the Janus membrane to act as unidirectional wound exudate inlets.

Channel layer patterning 314 may be performed on a biocompatible medical adhesive tape to define at least one wedge-shaped microfluidic channel (e.g., length approximately 2-8 mm, minimum width approximately 0.1 mm, maximum width approximately 1.2 mm). The interior of the channel can be rendered hydrophilic with hydrophobic edges via masked O₂ plasma to enhance capillary transport toward the sensing reservoir.

Micropillar fabrication 306 may create a three-dimensional graded micropillar array by molding polydimethylsiloxane (PDMS) against a 3D-printed or lithographic mold to yield pillars of differing heights (e.g., approximately 0.2-0.8 mm). Surface energy of the pillars can be tuned by silica coating and/or oxygen plasma to generate capillary pressure gradients that direct fluid away from the sensing reservoir and promote refreshing.

Sensor modification 320 may functionalize the electrode sites to realize the multiplexed electrochemical sensors. For example, a nitric oxide sensor may be formed by immobilizing hemoglobin on a LEG/graphene-oxide interface with an optional Nafion overcoat at a working electrode. An oxygen sensor may utilize a printed/sintered gold working electrode operated voltammetrically. A hydrogen peroxide sensor may be produced by electrodepositing Prussian Blue and, in certain embodiments, a nickel hexacyanoferrate overlayer with a permselective membrane on its working electrode. A pH sensor may be prepared by electropolymerizing polyaniline on a noble metal underlayer for potentiometric readout, and a temperature sensor may be realized as a printed carbon meander. A reference electrode may be completed by depositing silver onto a printed gold seed, converting to Ag/AgCl, and applying a polyvinyl butyral/salt reference membrane.

Finally, the flexible microfluidic assembly 322 may be realized by laminating, in registration, the Janus membrane (inlet layer), the patterned wedge-channel transport layer, and the graded PDMS micropillar outlet layer over the sensor patch so that the working electrodes and reference electrode are exposed within a sensing reservoir supplied by the wedge channel. Edge sealing and alignment features may ensure robust stacking and coupling to the wireless electronics, yielding the wearable wound management system in which wound exudate can be collected through the Janus membrane, directed along the wedge-shaped channel, interrogated at electrochemical or temperature sensors (e.g., sensors 202, 204, 206, 208, and 210), and evacuated via the graded micropillars for continuous, refreshed measurements.

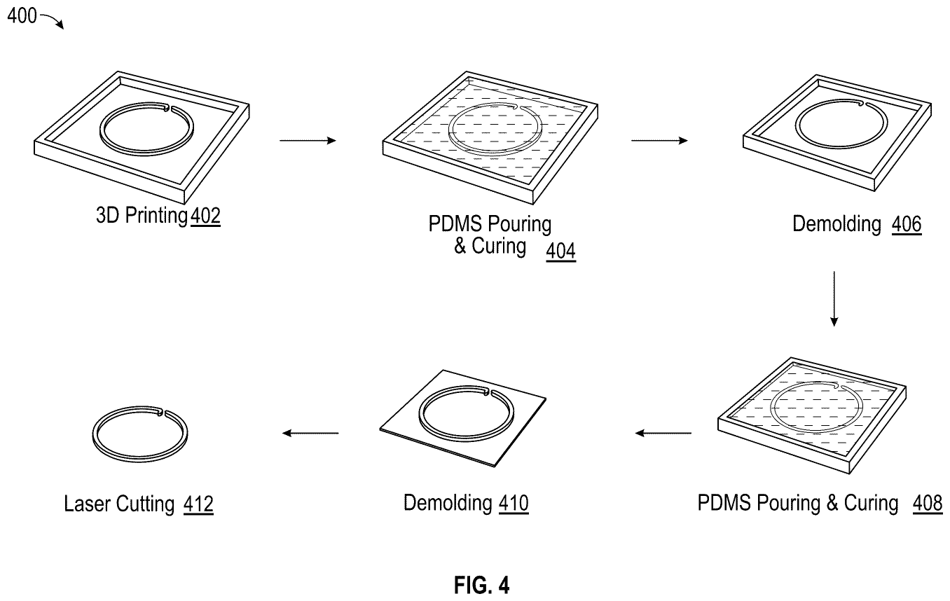

FIG. 4 illustrates an example fabrication process 400 for producing an array of micropillars that serves as the outlet/refreshing layer within the wearable wound management system. The process may yield a flexible polydimethylsiloxane (PDMS) structure featuring graded pillar heights and, in some embodiments, curved pillar profiles to generate capillary pressure gradients for unidirectional fluid transport.

The process may begin with 3D printing 402 of a master mold that defines the inverse geometry of the micropillar array. A high-resolution additive manufacturing method (e.g., SLA/DLP) may be used to realize pillar cavities with prescribed diameters, pitches, and a height gradient (for example, ranging from about 0.2 mm to about 0.8 mm). Curved or tapered cavity profiles may be incorporated to form curved-shaped pillars upon replication. The printed master can be post-cured and thermally conditioned to ensure dimensional stability and surface fidelity prior to molding.

Next, a first PDMS pouring and curing 404 may be performed by mixing PDMS prepolymer and curing agent (e.g., 10:1 ratio), degassing, and casting the mixture over the 3D-printed master to replicate its features. The PDMS may be cured (e.g., thermally) to form a solid elastomeric replica. First demolding 406 may separate the cured PDMS from the master, producing an intermediate negative replica that preserves the master's graded-height and curved feature set. This intermediate replica may act as a secondary mold to improve release and to invert the pattern for the final positive structure.

The second PDMS pouring and curing 408 may then be carried out by casting a fresh PDMS mixture onto the intermediate negative replica to generate the positive micropillar array. After curing under similar conditions, the second demolding 410 may yield the final PDMS sheet comprising the graded, curved-shaped pillars protruding from a flexible base. The resulting array may provide a built-in capillary pressure gradient configured to draw fluid vertically from an upstream wedge-shaped channel and laterally away from the sensing reservoir during operation.

Finally, laser cutting 412 may be used to dice the PDMS micropillar sheet to the desired outline, to open alignment holes or registration features, and to define any fluid windows or perimeter geometries required for subsequent lamination within the flexible microfluidic assembly. The completed micropillar array may be prepared for integration with the Janus membrane and channel layers over the sensor patch in the wearable wound management system.

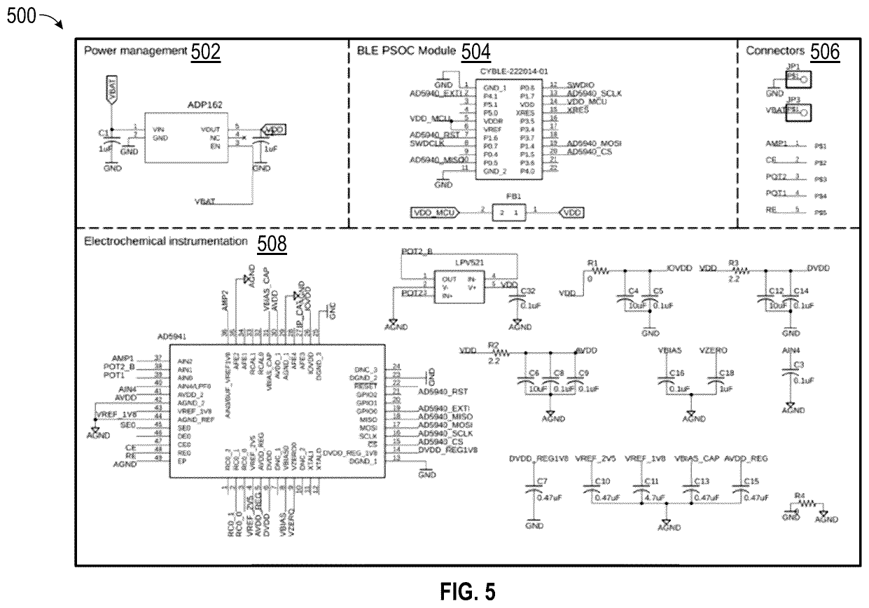

FIG. 5 depicts an example circuit diagram 500 for an example wearable wound management system. The electronics may be organized into functional blocks including power management 502, a BLE PSOC module 504 for control and wireless communication, connectors 506 to the flexible sensor patch, and electrochemical instrumentation 508 that implements potentiostat and sensing functions. These blocks may cooperate to bias the electrodes, acquire electrochemical and environmental signals, and transmit data wirelessly while operating from a compact battery supply.

Power management 502 may receive VBAT from a thin rechargeable or primary cell and generate regulated rails for the analog and digital domains using a low noise regulator. This block may include decoupling capacitors collocated at each rail, optional battery measurement (via divider to an ADC input), an enable pin for system on/off control, and ESD/transient protection on external interfaces. In certain embodiments, separate LDO outputs or filters are used to isolate the analog front end from digital switching noise.

The BLE PSOC module 504 may comprise a programmable system on chip with integrated Bluetooth Low Energy radio. The module may also provide: SPI (and/or I2C) control and data links to the electrochemical instrumentation 508; GPIOs for AFE reset/interrupt and measurement mode control; ADC channels for auxiliary sensors (e.g., resistive temperature sensor 202) and battery voltage; nonvolatile storage for calibration factors and BLE advertising/services for data streaming; firmware schedules measurements (e.g., differential pulse voltammetry, amperometry, potentiometry), applies temperature/pH compensation, and packages readings for wireless transmission.

Connectors 506 may provide the electrical interface to the sensor patch. In the illustrated embodiment, discrete pads or board to flex connectors may route the electrode nets, working electrode lines (e.g., for nitric oxide sensor, oxygen sensor, hydrogen peroxide sensor), a shared reference electrode 214 (RE), and a counter electrode (CE), to the electrochemical instrumentation 508. Additional pins may route the pH sensor and temperature sensor signals where read by the PSOC ADC. Mechanical alignment features may ensure reliable lamination and strain relief between the rigid PCB and flexible sensor.

Electrochemical instrumentation 508 may implement a multi-channel potentiostat and signal-conditioning chain suitable for the multiplexed sensors. In some embodiments, a single-chip analog front end (AFE), provides on-chip DAC biasing, a transimpedance amplifier with selectable feedback for current measurement, waveform generation for voltammetry, and an ADC for digitization. Low-noise op-amp buffers (e.g., for high-impedance pH electrodes) can be included to level-shift or filter signals prior to conversion. The AFE may control the potential between working electrodes and reference electrode while sourcing current through CE, measures faradaic currents from the electrochemical sensors during DPV and amperometry and acquires the open-circuit potential of pH sensor. Guarding, star-grounding between analog and digital returns, and local decoupling capacitors may be employed to minimize noise and drift.

Together, power management 502, BLE PSOC module 504, connectors 506, and electrochemical instrumentation 508 may form a compact, low-power electronics stack that biases and reads the electrochemical sensors on the sensor patch, compensates with temperature and pH, and wirelessly transmits wound biomarker data for processing and display.

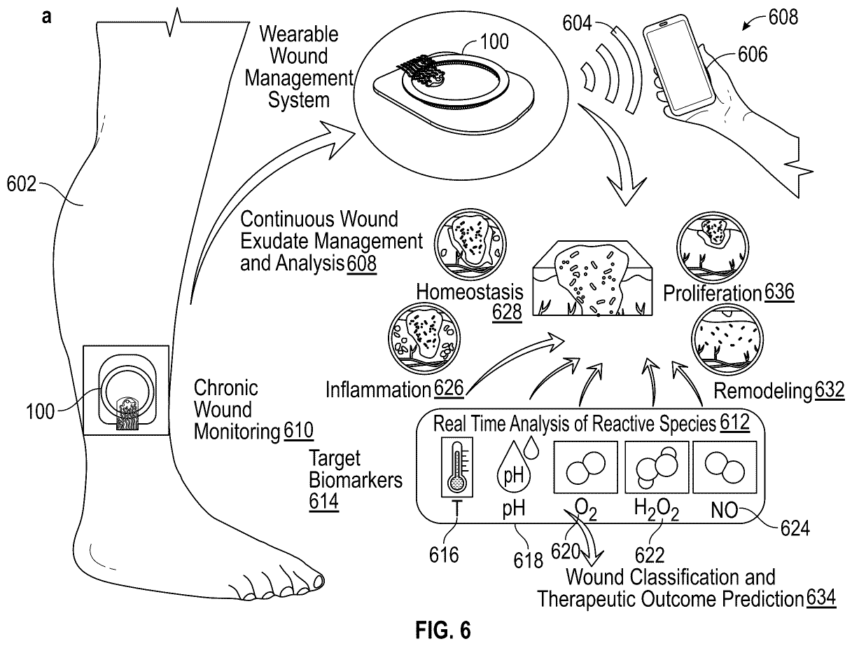

FIG. 6 illustrates a method for wearable wound exudate collection and analysis implemented on a human patient 602 using the wearable wound management system 100. In operation, wireless monitoring 604 may stream data from the on-body device to a provider or other person 606 for review, enabling continuous wound exudate management and analysis 608 and chronic wound monitoring 610 outside traditional clinical settings.

In embodiments, the method may include mounting the flexible microfluidic assembly over a wound to initiate continuous sampling and sensing. The assembly may passively collect exudate via the Janus membrane, transport the fluid through a wedge-shaped channel to a sensing region, and refreshes the sensing region through a graded micropillar array as discussed herein, thereby maintaining a supply of recently secreted fluid for real time analysis of reactive species 612. The system may then measure target biomarkers 614 and environmental parameters by actuating a multiplexed electrochemical sensor set: temperature 616 via a resistive temperature sensor; pH 618 via a potentiometric electrode; oxygen 620 and nitric oxide 624 by differential pulse voltammetry; and hydrogen peroxide 622 by amperometry. The temperature 616 and pH 618 readings may be used to calibrate the reactive-species measurements to account for known temperature- and pH-dependent response shifts, grounded in the disclosed in vitro characterizations.

As data accrues, the method may relate biomarker dynamics to wound-healing phases, including inflammation 626, homeostasis 628, proliferation 630, and remodeling 632. For example, elevated temperature 616, alkaline pH 618, and increased hydrogen peroxide 622 can signal heightened inflammation 626 or early infection, while increased oxygen 620 and sustained nitric oxide 624 may indicate improved perfusion and angiogenesis during proliferation 630. Conversely, decreases in hydrogen peroxide burden after intervention can reflect a transition toward homeostasis 628 and eventual remodeling 632. These associations may be grounded in practical research, which can demonstrate selective, stable in situ sensing of oxygen 620, nitric oxide 624, and hydrogen peroxide 622, along with validation against a clinical oxygen imaging benchmark.

The method may further comprise wireless monitoring 604 by transmitting processed measurements to a gateway or mobile device, where they are accessible to a provider or other person 606. The transmitted data may be analyzed to support wound classification and therapeutic outcome prediction 634, for instance by applying machine-learning models that use combinations of the target biomarkers 614 with contextual parameters to predict wound class or time to closure and to inform timely therapy adjustments. The method may enable continuous wound exudate management and analysis 608 and chronic wound monitoring 610 in human patients 602, providing actionable, real-time insight into wound status and trajectory based on temperature 616, pH 618, oxygen 620, hydrogen peroxide 622, and nitric oxide 624.

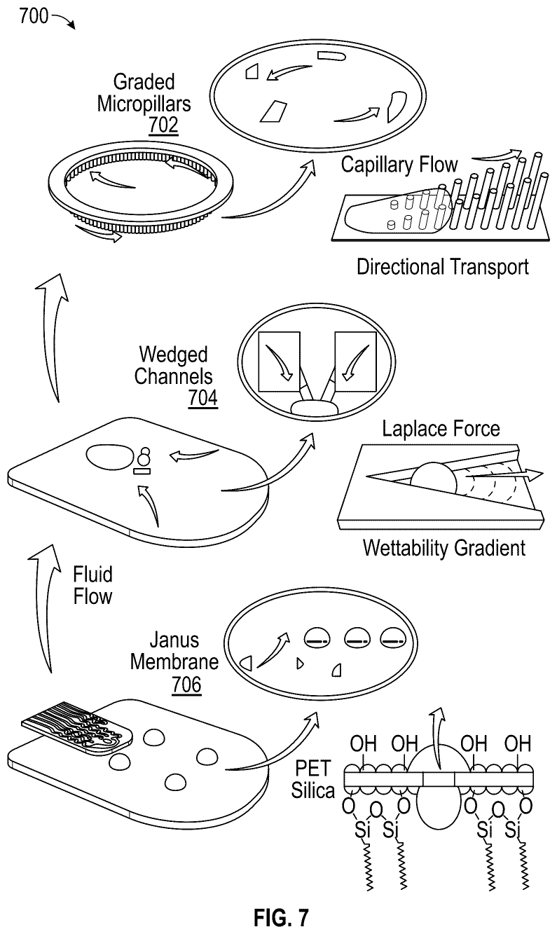

FIG. 7 illustrates an example flexible microfluidic assembly 700 that integrates a Janus membrane 706, wedged channels 704, and graded micropillars 702 to enable passive, pump-free fluid handling of wound exudate. The stack may be supported on a flexible PET base layer to maintain conformal skin contact while preserving structural integrity during body motion.

At the inlet, the Janus membrane 706 may be formed on a PET film bearing a silica coating that imparts opposing wetting properties: the wound-facing surface may be superhydrophobic and the opposing surface may be superhydrophilic. In certain embodiment, a perfluoroalkyl-functionalized silica layer may provide the superhydrophobicity, and O₂ plasma treatment of the opposite PET surface may yields superhydrophilicity. Laser-patterned inlets or micropores through the membrane may present a high wetting contrast across the film thickness, creating a curvature-driven pressure gradient (LaPlace force, e.g., Laplace pressure) that enables unidirectional fluid flow from the wound side to the device side without external pumping.

Downstream of the Janus membrane 706, the wedged channels 704 may be defined in a thin, biocompatible adhesive layer laminated to the PET. Each channel narrows near the inlet and expands toward a sensing region, establishing a capillary pressure gradient that automatically pulls fluid forward (e.g., fluid flow). This wedge geometry, inspired by cactus spines, may promote rapid fluid flow and efficient transport of small exudate volumes along the channel to the sensing region.

Beyond the sensing region, the graded micropillars 702 may comprise a three-dimensional PDMS array with a programmed height gradient (e.g., shorter pillars proximate the reservoir and taller pillars downstream). The graded architecture may generate directional capillary forces that wick fluid vertically and laterally away from the sensing region, thereby refreshing the sensing volume and minimizing mixing between newly arrived and previously resident fluid. Surface energy of the micropillars can be tuned (e.g., by silica treatment and plasma activation) to strengthen capillary action and maintain consistent fluid flow across the array.

Together, the Janus membrane 706, wedged channels 704, and graded micropillars 702 form the flexible microfluidic assembly 700 on PET that exploits LaPlace force and engineered wettability/geometry to achieve continuous, passive collection, transport, and refreshing of wound exudate. This configuration may be compatible with the multiplexed electrochemical sensor patch described herein.

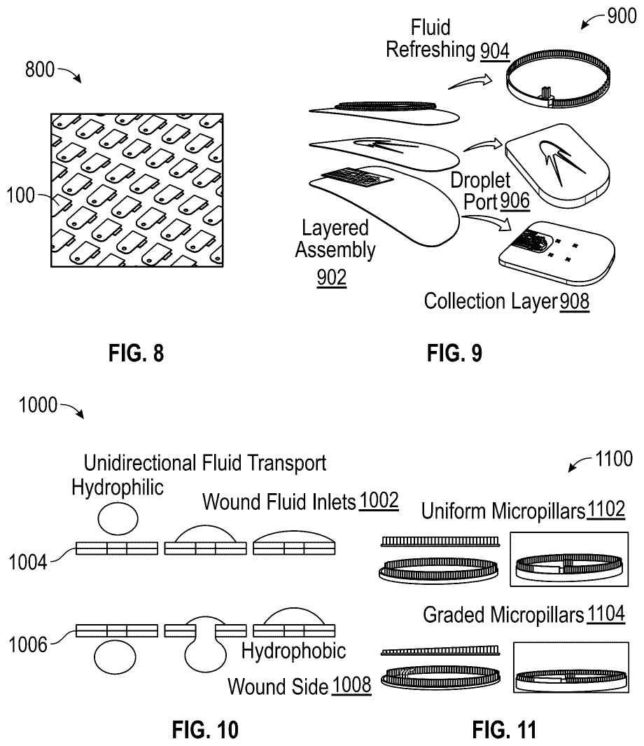

FIG. 8 depicts an array 800 of example wearable wound management systems 100 mass-produced via inkjet printing on a flexible polymer substrate. In the illustrated panelized layout, multiple identical device patterns may be printed in parallel using drop-on-demand inkjet deposition of conductive inks (e.g., gold and carbon) to define electrodes, interconnects/terminals, contact pads, and resistive elements on a PET sheet. The printed features may be subsequently cured to achieve target conductivity and adhesion, enabling high-throughput, uniform fabrication consistent with the scalable methods described herein.

The array 800 may incorporate registration fiducials and alignment features to facilitate downstream lamination of the microfluidic layers (including the Janus membrane, wedge-shaped transport channels, and graded micropillar array) and attachment of the wireless electronics. After printing and curing, apertures and sensing windows can be opened by CO₂ laser patterning, and the panel may be singulated by laser cutting or die cutting to yield individual wearable wound management systems 100. This panelized, inkjet-printed approach supports rapid, low-cost production of many units per sheet and is compatible with roll-to-roll processing for large-scale manufacturing.

FIG. 9 presents an expanded (exploded) view of an example flexible microfluidic assembly 900 configured as a layered assembly 902 for passive handling of wound exudate and delivery of refreshed samples to an underlying sensor patch. The assembly may include a droplet port 906, a collection layer 908 incorporating a Janus membrane, a transport layer with wedge-shaped channels defining a sensing reservoir, and an outlet layer comprising graded micropillars that provide fluid refreshing 904.

In the illustrated configuration, the droplet port 906 may be an opening in the collection layer 908. During use, wound exudate emerging from the wound bed, or, in bench testing, dispensed droplets, may enter through the droplet port 906 and contact the collection layer 908. The collection layer 908 may include a Janus membrane formed on a flexible film (e.g., PET) with opposing wetting properties, such that the wound-facing side is superhydrophobic while the opposing side is superhydrophilic. Laser-patterned micropores in the Janus membrane and the high wetting contrast may promote unidirectional transfer of small exudate volumes into the assembly, preventing backflow to the wound.

After the collection layer 908, a transport layer may define wedge-shaped channels that guide the incoming wound exudate to a sensing region aligned over the electrochemical sensor patch. The wedge geometry may establish a capillary pressure gradient to draw fluid forward, enabling reliable transport of low secretion rates to the sensing region. Downstream of the sensing region, an outlet layer of graded micropillars provides fluid refreshing 904. The graded micropillars may be molded (e.g., in PDMS) with a programmed height gradient to generate directional capillary forces that wick previously resident fluid vertically and laterally away from the reservoir, thereby minimizing mixing and maintaining a supply of newly arrived exudate over the sensors.

As assembled, the flexible microfluidic assembly 900 may use the layered assembly 902 (comprising the droplet port 906, the collection layer 908 with Janus membrane, the wedge-shaped transport geometry, and the graded micropillars effecting fluid refreshing 904) to continuously collect, route, and refresh wound exudate without pumps, supporting accurate, time-resolved in situ analysis when laminated to the multiplexed electrochemical sensor patch and wireless electronics described herein.

FIG. 10 illustrates an exemplary Janus membrane 1000 configured to admit wound exudate from a wound site 1008 through a set of wound fluid inlets 1002 and to promote unidirectional transport into the device. The membrane presents opposing wetting faces: a hydrophobic surface 1006 oriented toward the wound and a hydrophilic surface 1004 oriented toward the device interior. In operation, exudate at the wound site 1008 contacts the hydrophobic surface 1006 and enters the laser-patterned micropores that define the wound fluid inlets 1002. The large wetting contrast across the membrane thickness establishes a curvature-dependent LaPlace pressure that drives fluid from the hydrophobic side to the hydrophilic side, thereby achieving one-way flow and suppressing backflow to the wound.

In embodiments, the Janus membrane may be formed on a PET film using a perfluoroalkyl-functionalized silica coating to create the wound-facing superhydrophobic surface 1006, while selective O2 plasma treatment renders the opposite face superhydrophilic to serve as the hydrophilic surface 1004. The wound fluid inlets 1002 may be defined as micropores through the PET/SiO₂ stack to couple incoming exudate directly to downstream microfluidic features. This construction may enable rapid wetting on the device side, minimizes fouling and tissue adhesion on the wound side, and functions as an effective passive check valve and coarse filter for cellular debris.

As part of the overall fluid path, the hydrophilic surface 1004 of the Janus membrane 1000 may feed the collected exudate into the device's transport channels and sensing region, ensuring that freshly secreted fluid is delivered for analysis while protecting the wound interface at the hydrophobic surface 1006.

FIG. 11 depicts a two example arrays of micropillars 1100 formed on a flexible elastomeric substrate (e.g., PDMS) and shown in multiple perspective views to highlight geometry and function. The figure contrasts uniform micropillars 1102, in which pillar height and cross-section are substantially constant across the field, with graded micropillars 1104, in which pillar height increases monotonically along the intended fluid-flow direction. In representative embodiments, pillar diameters and pitch are held constant while heights vary (e.g., from about 0.2 mm to about 0.8 mm) to establish a capillary pressure gradient; optional curved or tapered sidewalls can further assist vertical transfer and lateral wicking.

In use, the array of micropillars 1100 may be positioned downstream of the sensing region within the flexible microfluidic assembly to manage fluid refreshing. The uniform micropillars 1102 may provide isotropic capillary pathways suitable for general drainage, whereas the graded micropillars 1104 may generate directional capillary forces that preferentially draw previously resident fluid away from the sensing region, thereby minimizing mixing with newly delivered exudate and improving temporal resolution of sensor readouts. Surface energy of the pillar tops and sidewalls can be tuned (e.g., by silica coating and/or plasma activation) to promote rapid wetting and consistent wicking performance. Alignment edges or cutouts (not numbered) may be included for registration when laminating this structure into the overall device stack.

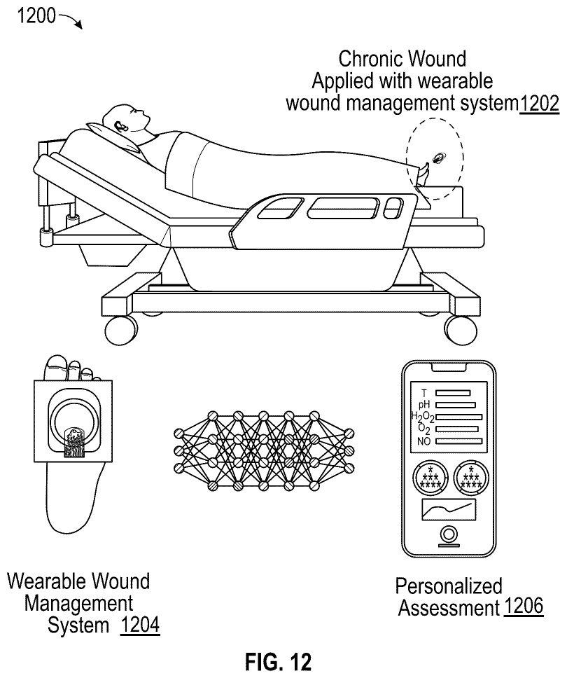

FIG. 12 illustrates an exemplary method of using an example wearable wound management system 1200 in a clinical setting to assess a chronic wound. In the depicted workflow, a chronic wound applied with wearable wound management system 1202 may be prepared at bedside, and a wearable wound management system applied to a patient 1204 may be shown secured over the wound. In representative use, the system may be sterilized (e.g., UV exposure and alcohol wipe), aligned so the flexible microfluidic assembly overlies the wound bed, and affixed with a medical dressing to ensure conformal contact without impeding patient mobility.

Once applied, the device may passively handle wound exudate via its triad microfluidics: a Janus membrane admits exudate from the wound surface, wedged channels transport small volumes to a sensing region, and graded micropillars continuously refresh the sensing region. Concurrently, the multiplexed electrochemical sensor patch may measure reactive species and environmental parameters in real time, and the onboard electronics perform calibration using temperature and pH before wirelessly streaming data for review.

The resulting data supports personalized assessment 1206 at the point of care and between visits. For example, elevations in temperature and pH with increased hydrogen peroxide can provide early indication of inflammation or incipient infection, while increased oxygen and sustained nitric oxide after vascular intervention may indicate improved perfusion and angiogenesis. In some embodiments, machine-learning models operating on the transmitted sensor features and basic patient information may generate objective wound classification and therapeutic outcome predictions (e.g., estimated healing time), enabling clinicians to tailor dressings, debridement schedules, antibiotics, or revascularization strategies based on the patient's real-time wound status.

In certain embodiments, the wearable wound management system may integrate an end-to-end machine-learning (ML) pipeline that transforms raw, multiplexed sensor signals into clinically actionable outputs. The device continuously collects wound exudate via a passive microfluidic assembly and acquires synchronized electrochemical measurements of target biomarkers (oxygen, nitric oxide, hydrogen peroxide) together with environmental parameters (pH, temperature). On-board calibration compensates reactive-species measurements using concurrently measured pH and temperature to account for known response dependencies. Motion artifacts and transient anomalies can be attenuated through filtering and quality checks prior to feature extraction.

Preprocessing produces standardized features from time-stamped measurements. In some embodiments, features include summary statistics (e.g., mean levels over a defined observation window), trends (e.g., short-term slopes or deltas before/after a clinical intervention), and stability metrics (e.g., variance or coefficient of variation). Objective patient attributes, such as age, presence of diabetes or peripheral artery disease (PAD), and wound area, may be appended to the feature vector when available. The system can operate with minimal inputs; for first-visit use, chemically derived features and objective metadata (age, PAD, diabetes) enable predictions without reliance on subjective clinical scoring or visit history.

Model training and selection can be performed using a portfolio of supervised learners. For example, linear, radial-basis, and sigmoid-kernel support vector classifiers (SVCs) and k-nearest neighbors (KNN) models may be utilized, with a linear SVC often selected based on validation accuracy and generalization. Wound-depth estimation was also possible using a compact feature set (e.g., nitric oxide, hydrogen peroxide, diabetes status, age). These embodiments illustrate, without limitation, that the pipeline supports multiple clinical inference tasks from the same multimodal signal stream.

Model explainability is supported through post hoc attribution methods such as Shapley additive explanations (SHAP). In embodiments, SHAP analysis can identify diabetes status, oxygen, and hydrogen peroxide as prominent contributors to wound classification, while PAD, wound area, pH, and nitric oxide ranked highly for healing-time prediction. Such interpretability assists clinician adoption, facilitates regulatory review, and enables automated rule checks (e.g., suppressing alerts when predictions are driven by low-confidence or out-of-bounds sensor inputs).

The ML pipeline also benefits from objective calibration and external validation. Dissolved oxygen readings from the wearable were validated against a United States FDA-cleared imaging system (Kent Imaging), yielding a Pearson correlation of approximately 0.943, which supports the fidelity of the input signals used for learning and inference. The system captured physiologically plausible trends around clinical interventions (e.g., increases in oxygen and nitric oxide following vascular procedures), and detected early infection signatures in preclinical continuous recordings (elevated temperature, pH, and hydrogen peroxide prior to visible signs), enabling timely escalation of care.

Deployment options include on-device inferencing, cloud-based processing, or hybrid schemes. In some embodiments, the device streams feature via Bluetooth Low Energy to a secure application, where models execute and return risk scores, wound class labels, or estimated healing times. The system may maintain rolling confidence indices, trigger alerts when probabilities cross programmable thresholds, and support periodic model refresh as additional de-identified patient data become available. These approaches enable use in both resource-limited and advanced clinical settings without disrupting existing workflows.

Overall, the field faces a convergence of challenges involving efficient fluid management, accurate and selective biomarker detection, and seamless integration of sensor data into wearable, continuous monitoring systems. Addressing these issues requires a comprehensive understanding of wound physiology, innovative microfluidic designs, and robust sensor manufacturing techniques. By solving the problem of isolating fresh exudate and ensuring reliable in situ biochemical analysis, advancements in this domain have the potential to transform wound care, reduce the burden on healthcare resources, and significantly improve patient outcomes.

It should be understood that the various features, aspects and functionality described in one or more of the individual embodiments are not limited in their applicability to the particular embodiment with which they are described. Instead, they can be applied, alone or in various combinations, to one or more other embodiments, whether or not such embodiments are described and whether or not such features are presented as being a part of a described embodiment. Thus, the breadth and scope of the present application should not be limited by any of the above-described exemplary embodiments.

Terms and phrases used in this document, and variations thereof, unless otherwise expressly stated, should be construed as open ended as opposed to limiting. As examples of the foregoing, the term “including” should be read as meaning “including, without limitation” or the like. The term “example” is used to provide exemplary instances of the item in discussion, not an exhaustive or limiting list thereof. The terms “a” or “an” should be read as meaning “at least one,” “one or more” or the like; and adjectives such as “conventional,” “traditional,” “normal,” “standard,” “known.” Terms of similar meaning should not be construed as limiting the item described to a given time period or to an item available as of a given time. Instead, they should be read to encompass conventional, traditional, normal, or standard technologies that may be available or known now or at any time in the future. Where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “component” does not imply that the aspects or functionality described or claimed as part of the component are all configured in a common package. Indeed, any or all of the various aspects of a component, whether control logic or other components, can be combined in a single package or separately maintained and can further be distributed in multiple groupings or packages or across multiple locations.

The terms “approximately” and “substantially” are used to account for variations that may occur due to manufacturing tolerances, measurement inaccuracies, and other practical considerations in implementing the described technology. The term “approximately” refers to a value or range that is close to the stated value but allows for minor deviations, typically within ±10%, unless otherwise specified, that do not materially affect the function or purpose of the invention. Similarly, the term “substantially” is used to indicate that a particular feature, characteristic, or result is largely present or achieved, with allowable variations without deviating from the intended scope and function of the invention. These terms should be interpreted in a manner consistent with the understanding of a person skilled in the art.

It should be noted that the terms “optimize,” “optimal” and the like as used herein can be used to mean making or achieving performance as effective or perfect as possible. However, as one of ordinary skill in the art reading this document will recognize, perfection cannot always be achieved. Accordingly, these terms can also encompass making or achieving performance as good or effective as possible or practical under the given circumstances, or making or achieving performance better than that which can be achieved with other settings or parameters.

Additionally, the various embodiments set forth herein are described in terms of exemplary block diagrams, flow charts, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A wearable wound management system, comprising:
a Janus membrane comprising a wound-facing superhydrophobic surface and an opposing superhydrophilic surface;
a wedge-shaped microfluidic channel in fluid communication with the Janus membrane;
an array of micropillars, wherein the array of micropillars comprises curved-shaped pillars capable of transferring fluid vertically from the wedge-shaped microfluidic channel to an upper gradient pillar array and a gradient of pillar heights arranged downstream of the wedge-shaped microfluidic channel;
a sensor patch laminated to the wedge-shaped microfluidic channel, the sensor patch comprising a flexible substrate and a plurality of electrochemical sensors printed thereon; and
a wireless electronic module electrically coupled to the sensor patch;
wherein the Janus membrane, the wedge-shaped microfluidic channel, the array of micropillars, the sensor patch, and the wireless electronic module define a flexible microfluidic assembly.

2. The wearable wound management system of claim 1, wherein the Janus membrane is formed by selective deposition of chemically modified silica nanoparticles and O₂ plasma etching on a polyethylene-terephthalate film.

3. The wearable wound management system of claim 2, wherein the chemical modified silica nanoparticles are 1H,1H,2H,2H-perfluorooctyltriethoxysilane-coated silica nanoparticles.

4. The wearable wound management system of claim 1, wherein the plurality of electrochemical sensors are selected from the list consisting of: a voltammetric nitric oxide sensor, a voltammetric oxygen sensor, an amperometric hydrogen peroxide sensor, a potentiometric pH sensor, and a resistive temperature sensor.

5. The wearable wound management system of claim 1, wherein the wedge-shaped microfluidic channel comprises a width that increases in a direction away from the Janus membrane and further comprises a curvature configured to increase capillary pressure.

6. The wearable wound management system of claim 1, wherein the array of micropillars comprises pillars fabricated from cured polydimethylsiloxane (PDMS) with heights ranging from 0.2 mm to 0.8 mm.

7. The wearable wound management system of claim 1, wherein the wireless electronic module is encapsulated in a biocompatible elastomer and is configured to transmit electrochemical sensor data via BLUETOOTH.

8. The wearable wound management system of claim 1, wherein the sensor patch is manufactured by drop-on-demand inkjet printing of conductive inks followed by CO₂ laser patterning.

9. A microfluidic module for wearable wound exudate handling, comprising:
an inlet layer comprising a Janus membrane comprising laser-patterned micropores and opposing hydrophobic/hydrophilic surfaces;
a transport layer comprising at least one wedge-shaped microfluidic channel defined in a biocompatible tape, the transport layer being in fluid communication with the inlet layer; and
an outlet layer comprising a three-dimensional graded micropillar array molded in polydimethylsiloxane, the outlet layer being in fluid communication with the transport layer.

10. The microfluidic module for wearable wound exudate handling of claim 9, wherein the Janus membrane comprises opposing surfaces including a superhydrophobic surface and a superhydrophilic surface, wherein the superhydrophobic surface comprises perfluoroalkyl-functionalized silica coating.

11. The microfluidic module for wearable wound exudate handling of claim 9, wherein the wedge-shaped channel in a range of 2-8 mm in length, a maximum width of approximately 0.8 mm, and a minimum width of approximately 0.1 mm.

12. The microfluidic module for wearable wound exudate handling of claim 9, wherein the three-dimensional graded micropillar array forms capillary pressure gradients configured to direct fluid away from the outlet layer.

13. The microfluidic module for wearable wound exudate handling of claim 9, further comprising an alignment feature for lamination to a flexible sensor substrate.

14. The microfluidic module for wearable wound exudate handling of claim 9, wherein the three-dimensional graded micropillar array comprises pillars of at least three discrete heights.

15. The microfluidic module for wearable wound exudate handling of claim 9, wherein the transport layer comprises a hydrophilic channel inside and hydrophobic edges defined by O₂ plasma etching.

16. A method for wearable wound exudate collection and analysis, comprising:
mounting a wearable device on a wound site, the wearable device comprising a microfluidic module stacked with a flexible electrochemical sensor patch and a wireless electronics module;
receiving wound exudate through a Janus membrane within the microfluidic module;
transporting the wound exudate from the microfluidic module through a wedge-shaped channel to a sensing reservoir;
refreshing the sensing reservoir by passing the wound exudate through a graded micropillar array;
electrochemically sensing the wound exudate to measure at least one reactive species and one environmental parameter via the electrochemical sensor patch; and
wirelessly transmitting measurement data representative of electrochemically sensing the wound exudate with the wireless electronics module.

17. The method of claim 16, wherein electrochemically sensing the wound exudate further comprises characterizing nitric oxide, oxygen, and hydrogen peroxide concentrations by differential pulse voltammetry and amperometry.

18. The method of claim 16, further comprising calibrating the flexible electrochemical sensor patch using sensed pH and temperature.

19. The method of claim 16, wherein refreshing the sensing reservoir comprises unidirectional fluid movement out of the reservoir via the graded micropillar array.

20. The method of claim 16, further comprising analyzing the transmitted measurement data with a machine-learning model to predict wound classification or healing time.
